(19) United States
(12) Patent Application Publication
(10) Pub. No.: US 2025/0177608 A1
(43) Pub. Date: Jun. 5, 2025

(54) COATING FORMULATION

(71) Applicant: RUHR UNIVERSITY BOCHUM, Bochum (DE)

(72) Inventors: Kenneth Wiliam DALGARNO, Newcastle upon Tyne (GB); Alexander SIEBERATH, Bochum (DE); Piergiorgio GENTILE, Newcastle upon Tyne (GB); Ana FERREIRA-DUARTE, Newcastle upon Tyne (GB)

(21) Appl. No.: 18/845,151

(22) PCT Filed: Mar. 10, 2023
(86) PCT No.: PCT/GB2023/050571

§ 371 Date: Sep. 9, 2024

(30) Foreign Application Priority Data
Mar. 10, 2022 (GB) 2203317.9

(51) Int. Cl. A61L 27/34; A61L 27/32; C12N 5/077

(52) U.S. Cl. CPC A61L 27/34; A61L 27/32; C12N 5/0654; A61L 2400/18; A61L 2420/02; A61L 2420/08; C12N 2533/18; C12N 2533/54

(57) ABSTRACT

The present invention relates to formulations, comprising SBF and collagen, and methods that form a bone-like coating on the surface of a substrate. These coated substrates can be used in cell culture experiments to investigate the bone remodelling process and to evaluate drugs for diseases related to bone remodelling, e.g. osteoporosis.

This invention relates to a formulation that can be used to deposit a bone-like coating onto a substrate, e.g. a polymer substrate, and a method of depositing the same. The bone-like coating is capable of supporting cell cultures.

### BACKGROUND

Bone is made primarily of calcium phosphate hydroxyapatite interspersed in a collagen matrix. A dynamic organ, bone is constantly remodeled by bone-forming osteoblasts and bone-resorbing osteoclasts. Bone resorption is the process by which osteoclasts break down bone and release inorganic minerals, mainly calcium ions, and organic components, which is mostly degraded collagen fragments, to the extracellular space. Osteoclasts are the cells responsible for resorbing both the organic and inorganic components of bone.

An imbalance in the function of these cell types can causes bone related diseases such as osteoporosis. Osteoporosis affects over 3 million people in the UK and more than 500,000 people receive hospital treatment for fragility fractures (bones that break after falling from standing height or less) every year as a result of osteoporosis. Since many bone diseases such as osteoporosis are due to increased bone resorption, the osteoclast is a main target for the pharmaceutical industry to find therapeutic solutions for bone diseases.

In order to effectively assay pharmaceutical compounds against the treatment and/or prevention of bone related diseases and disorders such as osteoporosis, there is therefore a need to simulate the natural bone environment. Compounds can then be assayed within this environment and their effects on the bone remodelling process can be studied.

Simulated body fluid (SBF) solutions have been used to induce apatitic calcium phosphate formation on metals, ceramics, and polymers. SBF solutions are prepared with the aim of simulating the ion concentrations present in human plasma. A. C. Tas and S. B. Bhaduri, J. Mater. Res., 2004, 19, 2742-2749 reported the utilization of 10×SBF to deposit bone-like apatitic calcium phosphate on Ti6Al4V. WO 2011/149741 (A1) discloses substrates composed of a fluorescently labelled calcium phosphate coating on a base prepared using calcein mixed with 5×SBF. Y. Chen, et al., J. Biomed. Mater. Res. B. Appl. Biomater., 2006, 77, 315-322 discloses collagen and apatite coprecipitated as a composite coating on poly L-lactic acid (PLLA) using an incubation solution containing collagen (1 g/L) and 5×SBF.

It is an aim of certain embodiments of the present invention to provide compositions and methods that form a bone-like coating on the surface of a substrate. These coated surfaces can then be used in cell culture experiments to investigate the bone remodelling process and to evaluate drugs for diseases related to bone remodelling, e.g. osteoporosis.

### BRIEF SUMMARY OF THE DISCLOSURE

### Coating Formulation

In accordance with a first aspect of the invention is provided a coating formulation comprising simulated body fluid (SBF) and collagen, wherein the concentration of Ca²⁺ in the formulation is ≥7.5 mM and the concentration of HPO₄²⁻ in the formulation is ≥3.0 mM.

It may be that the concentration of Ca²⁺ in the formulation is ≥12.5 mM. It may be that the concentration of HPO₄²⁻ in the composition is ≥5 mM. It may be that the concentration of Ca²⁺ in the formulation is >12.5 mM. It may be that the concentration of HPO₄²⁻ in the composition is >5 mM. It may be that the concentration of Ca²⁺ in the formulation is ≥15 mM. It may be that the concentration of HPO₄²⁻ in the composition is ≥6 mM. It may be that the concentration of Ca²⁺ in the composition is ≥18 mM. It may be that the concentration of HPO₄²⁻ in the composition is ≥7.5. mM. Preferably, the concentration of Ca²⁺ in the composition is ≥20.0 mM. Preferably, the concentration of HPO₄²⁻ in the composition is ≥8.0 mM.

It may be that the concentration of Ca²⁺ in the formulation is ≤42 mM. It may be that the concentration of HPO₄²⁻ in the formulation is ≤17 mM. It may be that the concentration of Ca²⁺ in the formulation is ≤31.5 m, e.g. ≤26 mM. It may be that the concentration of HPO₄²⁻ in the formulation is ≤12.5 mM, e.g. ≤10.5 mM.

Without wishing to be bound by theory, a bone like coating is thought to develop on the surface of substrate when in contact with the coating formulation through firstly, the nucleation of calcium phosphate on the surface followed by the interspersion of polymerised collagen throughout the nucleated calcium phosphate. It is conceived that through the selection of simulated body fluid ion and collagen concentrations in the coating formulation (e.g. Ca²⁺, HPO₄²⁻ and collagen concentration of ≥7.5 mM, ≥3.0 mM and ≥0.2 mg/mL, respectively) calcium phosphate nucleates on the surface of the substrate at a faster rate than the polymerisation of the collagen.

The SBF may also comprise Nat. The concentration of Na⁺ in the formulation may be in the range from 255 mM to 1273 mM. The SBF may also comprise Cl⁻. The concentration of Cl⁻ in the formulation may be in the range from 268 mM to 1342 mM. The SBF may also comprise Mg⁺. The concentration of Mg⁺ in the formulation may be in the range from 1.25 mM to 6.75 mM. The SBF may also comprise K⁺. The concentration of K⁺ in the formulation may be in the range from 1.25 mM to 6.75 mM.

The concentration of Nat in the formulation may be >505 mM, e.g. in the range from >505 mM to 1262 mM. The concentration of Cl in the formulation may be >532 mM, e.g. in the range from >532 mM to 1331 mM. The concentration of Mg⁺ in the formulation may be >2.5 mM, e.g. in the range from >2.5 mM to 6.25 mM. The concentration of K⁺ in the formulation may be >2.5 mM, e.g. in the range from >2.5 mM to 6.25 mM.

The collagen may be Type I collagen, e.g. atelocollagen. The collagen may be enzymatically cleaved collagen.

It may be that the concentration of collagen in the formulation is ≥0.2 mg/mL, e.g. ≥0.5 mg/mL. It may be that the concentration of collagen in the formulation is ≥0.8 mg/mL. It may be that the concentration of collagen in the formulation is in the range from 0.2 mg/mL to 5.0 mg/mL. It may be that the concentration of collagen in the formulation is in the range from 0.3 mg/ml to 1.5 mg/mL, e.g. 0.8 mg/mL to 1.2 mg/mL.

The concentration of collagen in the formulation may be in the range from 0.1 mg/mL to 0.9 mg/mL. It may be that the concentration of collagen in the formulation is in the range from 0.25 mg/mL to 0.75 mg/mL, e.g. 0.30 mg/mL to 0.70 mg/mL. It may be that the concentration of collagen in the formulation is in the range from 0.35 mg/ml to 0.65 mg/mL, e.g. 0.40 mg/mL to 0.60 mg/mL.

Although it has been found that with increasing collagen concentration (from 0.25 to 1 mg/mL), more collagen is incorporated in the coating, a collagen concentration of around 0.5 mg/mL has been shown to increase the deposition of calcium phosphate particles in comparison to a 1 mg/mL concentration.

It may be that the pH of the formulation is in the range from 6.1 to 6.6., e.g. 6.20 to 6.35. Preferably, the pH of the formulation is in the range from 6.25 to 6.30. The formulation may further comprise sodium bicarbonate. It may be that the formulation does not comprise a buffer.

### Method of Deposition

In accordance with a second aspect of the invention is provided a method for depositing a bone-like coating onto a substrate, the method comprising step c): contacting the substrate with a coating formulation according to the first aspect of the invention to form a coated substrate comprising a bone-like coating.

In embodiments, the method also comprises steps a) and b):
a) adding collagen to a simulated body fluid (SBF), wherein the concentration of Ca²⁺ in the SBF is ≥7.5 mM and the concentration of HPO₄²⁻ in the SBF is ≥3 mM, to form an intermediate formulation; and
b) adjusting the pH of the intermediate formulation to be in the range from 6.1 to 6.6, e.g. 6.20 to 6.35, to form the coating formulation.

Step a) may comprise adding a collagen solution to the SBF. The concentration of collagen in the collagen solution may be in the range from 4 mg/ml to 8 mg/ml, e.g. 5 mg/mL to 7 mg/mL.

Preferably, step b) comprises adjusting the pH of the intermediate formulation to be in the range from 6.25 to 6.30 to form the coating formulation. It may be that step b) is achieved by adding sodium bicarbonate to the intermediate formulation.

It may be that the concentration of Ca²⁺ in the SBF is ≥12.5 mM and the concentration of HPO₄²⁻ in the SBF is ≥5 mM. It may be that the concentration of Ca²⁺ in the SBF is >12.5 mM. It may be that the concentration of HPO₄²⁻ in the SBF is >5 mM. It may be that the concentration of Ca²⁺ in the SBF is ≥15 mM. It may be that the concentration of HPO₄²⁻ in the SBF is ≥6 mM. It may be that the concentration of Ca²⁺ in the SBF is ≥18.8 mM and the concentration of HPO₄²⁻ in the SBF is ≥7.5. mM. Preferably, the concentration of Ca²⁺ in the SBF is ≥22.5 mM and the concentration of HPO₄²⁻ in the composition is ≥9.0 mM.

It may be that the concentration of Ca²⁺ in the SBF is ≤50 mM. It may be that the concentration of HPO₄²⁻ in the SBF is ≤20 mM. It may be that the concentration of Ca²⁺ in the SBF is ≤37.5 m, e.g. ≤31 mM. It may be that the concentration of HPO₄²⁻ in the SBF is ≤15 mM, e.g. ≤12.5 mM.

The SBF may also comprise Nat. The concentration of Nat in the SBF may be in the range from 303 mM to 1515 mM. The SBF may also comprise Cl⁻. The concentration of Cl in the SBF may be in the range from 320 mM to 1598 mM. The SBF may also comprise Mg⁺. The concentration of Mg⁺ in the SBF may be in the range from 1.5 mM to 7.5 mM. The SBF may also comprise K⁺. The concentration of K⁺ in the SBF may be in the range from 1.5 mM to 7.5 mM.

The concentration of Nat in the SBF may be >505 mM, e.g. in the range from >505 mM to 1262 mM. The concentration of CI in the SBF may be >532 mM, e.g. in the range from >532 mM to 1331 mM. The concentration of Mg⁺ in the SBF may be >2.5 mM, e.g. in the range from >2.5 mM to 6.25 mM. The concentration of K⁺ in the SBF may be >2.5 mM, e.g. in the range from >2.5 mM to 6.25 mM.

The collagen may be as defined above for the first aspect of the invention.

It may be that the concentration of collagen in the intermediate and coating formulations is ≥0.2 mg/mL, e.g. ≥0.5 mg/mL. It may be that the concentration of collagen in the intermediate and coating formulations is ≥0.8 mg/mL. It may be that the concentration of collagen in the intermediate and coating formulations is in the range from 0.2 mg/mL to 5.0 mg/mL. It may be that the concentration of collagen in the intermediate and coating formulations is in the range from 0.3 mg/mL to 1.5 mg/ml, e.g. 0.8 mg/mL to 1.2 mg/mL.

The concentration of collagen in the intermediate and coating formulations may be in the range from 0.1 mg/mL to 0.9 mg/mL. It may be that the concentration of collagen in the intermediate and coating formulations is in the range from 0.25 mg/ml to 0.75 mg/mL, e.g. 0.30 mg/mL to 0.70 mg/mL. It may be that the concentration of collagen in the intermediate and coating formulations is in the range from 0.35 mg/ml to 0.65 mg/mL, e.g. 0.40 mg/ml to 0.60 mg/mL.

It may be that the pH of the SBF is in the range from 6.1 to 6.6, e.g. 6.20 to 6.35. It may be that the pH of SBF is in the range from 6.25 to 6.30. The SBF may comprise sodium bicarbonate.

Another possible use of the present invention is to deposit a bone-like coating onto a medical implant in order to increase bone regrowth and improve the fixation of the implant within the body. It may be therefore that the substrate is a metal substrate. The metal may be a metal alloy. The metal may be a metal or a metal alloy commonly used in medical implants. For example, the metal may be stainless steel, titanium, a titanium alloy, a cobalt chrome alloy, or tantalum.

In other embodiments, the substrate is a polymer substrate. It may be that the polymer is polyethylene (PE), polypropylene (PP), polyvinyl chloride (PVC), polystyrene (PS), a nylon (e.g. nylon 6 or nylon 6,6), polytetrafluoroethylene (PTFE), a thermoplastic polyurethane (TPU) or a polyacrylate. The polymer may be poly L-lactic acid (PLLA). The polymer may comprise ester groups. The polymer may comprise carboxylic acid groups. The carboxylic acid groups may have been formed by saponification of the ester groups in a polymer substrate precursor. The polymer substrate may be a polyacrylate. The polymer substrate may be a polyacrylate which has been activated by saponification of the ester groups, e.g. by exposure to hydroxide ions, e.g. NaOH. The polymer substrate may be tissue culture plastic (TCP).

The method may comprise activating the substrate, e.g. by exposing it to hydroxide ions, e.g. NaOH, before it is coated.

The aforementioned polymers may be formed into cell culture vessels, e.g. plates, wells, multi-well plates, and flasks.

It may be that the substrate is a cell culture plate. It may be that the substrate is a 3D printed object and/or a medical implant.

Step c) may comprise submerging the substrate in the coating formulation. It may be that step c) is carried out for at least 2 hours, e.g. at least 4 hours. It may be that step c) is carried out for at least 6 hours. It may be that step c) is carried out at a temperature in the range from 0° C. to 37° C. It may be that step c) is carried out at a temperature in the range from 15° C. to 30° C., e.g. at room temperature. Step c) may be carried out in an incubator. Where the substrate is a cell culture plate, it may be that step c) comprises submerging the cell culture plate in the coating formulation in an upright position. Alternatively, step c) may comprise submerging the cell culture plate in the coating formulation in an inverted position. Submerging the cell culture plate in an inverted position has been found to lead to a more homogenous deposition of the coating on the substrate surface since this avoids incorporation of precipitates into the coating.

After the coated substrate is formed, the coating formulation is removed from the coated substrate, or the coated substrate is removed from the coating formulation. The coated substrate may then be washed, e.g. with deionised water. Any excess liquid may be removed from the coated substrate. This may include drying the coated substrate, e.g. at room temperature. Where the substrate is a cell culture plate, this may include positioning the coated cell culture plate so that the well or wells of the culture plate face downwards.

The method may further comprise step d): incubating the coated substrate in a basal medium, such as Dulbecco's Modified Eagle's Medium (DMEM). It may be that step d) is carried out for at least 6 hours, e.g. at least 10 hours. It may be that step d) is carried out at a temperature in the range from 25° C. to 50° C. It may be that step c) is carried out at a temperature in the range from 34° C. to 40° C. Step c) may be carried out in an incubator. Incubation in a basal medium has been found to increase the stability of the coating on the surface of the substrate.

The method may further comprise fluorescently labelling the bone-like coating in order to form a fluorescently labelled bone-like coating. This may be achieved by contacting the bone-like coating with a fluorescent label. The fluorescent label may be a fluorophore or a fluorescent dye. The fluorescent label may be a calcium binding fluorescent label. The fluorescent label may be calcein, e.g. calcein green.

### Bone Remodelling Method

In accordance with a third aspect of the invention is provided a method for assessing bone remodelling, the method comprising steps i), ii) and iv):
i) carrying out a process according to the second aspect to form a coated substrate;
ii) culturing cells on the coated substrate to form a cell culture; and
iv) monitoring the bone-like coating and/or the bone remodelling activity of the cell culture.

The cells may be associated with bone remodelling. For example, the cells may comprise osteoblasts, osteocytes, osteoclasts, stem cells, endothelial cells, chondrocytes, or a combination thereof. The stem cells may be mesenchymal stem cells. The cells may comprise osteoclasts. Preferably, the cells comprise osteoblasts and osteoclasts.

Step ii) may comprise culturing cells in a culture medium on the coated substrate to form a cell culture. In a preferred embodiment, the plate is placed in a controlled culture environment for a period (e.g., 37° C., 5% CO₂ incubator for 2-4 hours) to allow for cell attachment.

The method may comprise differentiating stem cells, e.g. mesenchymal stem cells, to provide osteoblasts, osteocytes, osteoclasts, or a combination thereof.

Not wishing to be bound by theory, when certain cells (e.g. osteoclasts) come into contact with the bone-like coating, these cells will break down the bone-like coating forming calcium phosphate fragments which are released into the cell culture (resorption). Certain other cells (e.g. osteoblasts) will utilise these calcium phosphate fragments in order to re-form bone in or on the bone-like coating. Step iv) may therefore comprise assessing the resorption of the bone-like coating and the formation of bone.

To do this, the bone-like coating can be fluorescently labelled. Step ii) may therefore also comprise fluorescently labelling the bone-like coating (e.g. as described above for the second aspect). Step ii) may comprise culturing cells in a culture medium supplemented with a fluorescent label, e.g. calcein, on the coated substrate to form a cell culture and a fluorescently labelled bone-like coating. The calcein may be calcein green.

Again, not wishing to be bound by theory, when certain cells (e.g. osteoclasts) come into contact with a fluorescently labelled bone-like coating, these cells will break down the bone-like coating forming fluorescently labelled calcium phosphate fragments. Fluorescently labelled calcium phosphate fragments can be detected by methods known in the art for detecting the particular fluorescent label used. For example, if the bone-like coating is labelled with calcein, its fluorescence can be detected by the use of a fluorimeter with excitation and emission wavelengths of 495 and 515 nm, respectively.

The fluorescently labelled bone-like coating and cell culture may be imaged by phase contrast microscopy (PCM). Quantification of the resorption and formation of the bone-like coating can be achieved by using a high- and low-intensity fluorescence threshold on the captured image, the high-intensity threshold indicating the deposition of minerals by cells and the low-intensity threshold indicating the resorption of bone-like coating.

Step i) may further comprise sterilising the coated substrate, e.g. by UV sterilisation. Steps ii) and iv) may be carried out in an incubator. The method of the third aspect is capable of high-throughput, large-scale, automated applications. The method is particularly applicable to detecting and establishing unanticipated or unintended actions of drugs or drug candidates during drug discovery, development and approval (DDDA). The method of the third aspect is particularly suitable for establishing secondary therapeutic claims (“new uses” or “new indications”) and determining toxicities both of known compounds and new compounds.

Actions on a drug target, e.g. osteoclasts, can be evaluated against large numbers of compounds by use of high-throughput screening (HTS) assays that measure the activity or state of the target.

It may be therefore that the method of the third aspect is a method for assaying a pharmaceutical compound, the method further comprising step iii): contacting the cell culture with a pharmaceutical compound. In this method step iv) may also comprise measuring the cell viability.

The method of the third aspect may be a method of identifying a therapeutic agent, e.g. a pharmaceutical compound, for a bone disease, the method comprising steps i) to v):
i) carrying out a process according to the second aspect to form a coated substrate;
ii) culturing cells on the coated substrate to form a cell culture;
iii) contacting the cell culture with a therapeutic agent;
iv) monitoring the bone remodelling activity of the cell culture and/or measuring the cell viability; and
v) selecting a therapeutic agent based on the measured cell viability and/or bone remodelling activity.

The therapeutic agent, e.g. pharmaceutical compound may be for use in the treatment of a bone disease, e.g. osteoporosis. The activity of the agent can be assessed by, e.g. monitoring the affect this agent has on the fluorescence of a fluorescently labelled bone-like coating and/or the cell culture.

### Kit

In accordance with a fourth aspect of the invention is provided a kit comprising: simulated body fluid (SBF), wherein the concentration of Ca²⁺ in the SBF is ≥7.5 mM and the concentration of HPO₄²⁻ in the SBF is ≥3 mM; and a collagen solution.

It may be that the concentration of Ca²⁺ in the SBF is ≥12.5 mM and the concentration of HPO₄²⁻ in the SBF is ≥5 mM. It may be that the concentration of Ca²⁺ in the SBF is ≥12.5 mM. It may be that the concentration of HPO₄²⁻ in the SBF is ≥5 mM. It may be that the concentration of Ca²⁺ in the SBF is ≥15 mM. It may be that the concentration of HPO₄²⁻ in the SBF is ≥6 mM. It may be that the concentration of Ca²⁺ in the SBF is ≥18.8 mM and the concentration of HPO₄²⁻ in the SBF is ≥7.5. mM. Preferably, the concentration of Ca²⁺ in the SBF is ≥22.5 mM and the concentration of HPO₄²⁻ in the SBF is ≥9.0 mM.

It may be that the concentration of Ca²⁺ in the SBF is ≤50 mM. It may be that the concentration of HPO₄²⁻ in the SBF is ≤20 mM. It may be that the concentration of Ca²⁺ in the SBF is ≤37.5 m, e.g. ≤31 mM. It may be that the concentration of HPO₄²⁻ in the SBF is ≤15 mM, e.g. ≤12.5 mM.

The SBF may also comprise Nat. The concentration of Nat in the SBF may be in the range from 303 mM to 1515 mM. The SBF may also comprise Cl⁻. The concentration of Cl in the SBF may be in the range from 320 mM to 1598 mM. The SBF may also comprise Mg⁺. The concentration of Mg⁺ in the SBF may be in the range from 1.5 mM to 7.5 mM. The SBF may also comprise K⁺. The concentration of K⁺ in the SBF may be in the range from 1.5 mM to 7.5 mM.

The concentration of Na⁺ in the SBF may be >505 mM, e.g. in the range from >505 mM to 1262 mM. The concentration of Cl in the SBF may be >532 mM, e.g. in the range from >532 mM to 1331 mM. The concentration of Mg⁺ in the SBF may be >2.5 mM, e.g. in the range from >2.5 mM to 6.25 mM. The concentration of K⁺ in the SBF may be >2.5 mM, e.g. in the range from >2.5 mM to 6.25 mM.

The concentration of collagen in the collagen solution may be in the range from 4 mg/mL to 8 mg/mL, e.g. 5 mg/mL to 7 mg/mL. The collagen may be as defined above for the first aspect of the invention.

The kit may further comprise sodium bicarbonate. The kit may comprise a cell culture plate.

### Coated Substrate

In accordance with a fifth aspect of the invention is provided a coated substrate. The coated substrate may comprise:
a substrate; and
a coating on the substrate, wherein the coating comprises calcium phosphate at the interface between the substrate and the coating and collagen deposited on the calcium phosphate.

It may be that the calcium phosphate is dispersed within the collagen.

The substrate may be as defined above for the second aspect of the invention.

The calcium phosphate may comprise hydroxyapatite, e.g. amorphous carbonated hydroxyapatite.

The coating may be fluorescently labelled with a fluorescent label as described above for the second aspect.

It may be that the coated substrate is obtainable by a method according to the second aspect of the invention. It may be that the coated substrate is obtained by a method according to the second aspect of the invention.

The coated substrate of the fifth aspect may be useful in a screening assay, e.g. a high-throughput screening assay. Therefore, according to a sixth aspect of the invention is provided the use of the coated substrate according to the fifth aspect in a screening assay, e.g. a high-throughput screening assay. The assay may be to identify a therapeutic agent for a bone disease, e.g. osteoporosis.

The coated substrate of the fifth aspect may be useful in an invitro osteoporosis model. Therefore, according to a seventh aspect of the invention is provided the use of the coated substrate according to the fifth aspect in an invitro osteoporosis model.

The invention may be as described in any of the following numbered paragraphs:
1. A coating formulation comprising simulated body fluid (SBF) and collagen, wherein the concentration of Ca²⁺ in the formulation is ≥7.5 mM and the concentration of HPO₄²⁻ in the formulation is ≥3.0 mM.
2. A formulation according to paragraph 1, wherein the concentration of Ca²⁺ in the formulation is ≥18.8 mM and the concentration of HPO₄²⁻ in the formulation is ≥7.5 mM.
3. A formulation according to paragraph 1 or paragraph 2, wherein the concentration of collagen in the formulation is in the range from 0.1 mg/ml to 5.0 mg/mL.
4. A formulation according to paragraph 3, wherein the concentration of collagen in the formulation is in the range from 0.3 mg/ml to 1.2 mg/mL.
5. A formulation according to any preceding paragraph, wherein the pH of the formulation is in the range from 6.1 to 6.6.
6. A formulation according to paragraph 5, wherein the pH of the formulation is in the range from 6.25 to 6.30.
7. A formulation according to any preceding paragraph, wherein the formulation further comprises sodium bicarbonate.
8. A method for depositing a bone-like coating onto a substrate, the method comprising step c): contacting the substrate with a coating formulation according to any one of paragraphs 1 to 7 to form a coated substrate.
9. A method according to paragraph 8, wherein the method also comprises steps a) and b):
   a) adding collagen to a simulated body fluid (SBF), wherein the concentration of Ca²⁺ in the SBF is ≥7.5 mM and the concentration of HPO₄²⁻ in the SBF is ≥3 mM, to form an intermediate formulation; and
   b) adjusting the pH of the intermediate formulation to be in the range from 6.1 to 6.6 to form the coating formulation.
10. A method according to paragraph 9, wherein step a) comprises adding a collagen solution to the simulated body fluid (SBF).
11. A method according to paragraph 10, wherein the concentration of collagen in the collagen solution is in the range from 4 mg/ml to 8 mg/mL.
12. A method according to any one of paragraphs 9 to 11, wherein step b) comprises adjusting the pH of the intermediate formulation to be in the range from 6.25 to 6.30.
13. A method according to any one of paragraphs 9 to 12, wherein step b) comprises adding sodium bicarbonate to the intermediate formulation.
14. A method according to any one of paragraphs 9 to 13, wherein the concentration of Ca²⁺ in the SBF is ≥18.8 mM and the concentration of HPO₄²⁻ in the SBF is ≥7.5 mM.
15. A method according to any one of paragraphs 9 to 14, wherein the pH of the SBF is in the range from 6.1 to 6.6.
16. A method according to any one of paragraphs 8 to 15, wherein the substrate is a metal substrate.
17. A method according to any one of paragraphs 8 to 15, wherein the substrate is a polymer substrate.
18. A method according to paragraph 17, wherein the substrate is a cell culture plate.
19. A method according to any one of paragraphs 8 to 17, wherein the substrate is a 3D printed object and/or a medical implant.
20. A method for assessing bone remodelling, the method comprising steps i), ii) and iv):
   i) carrying out the process of any one of paragraphs 8 to 19 to form a coated substrate;
   ii) culturing cells on the coated substrate to form a cell culture; and
   iv) monitoring the bone-like coating and/or the bone remodelling activity of the cell culture.
21. A method according to paragraph 20, wherein the cells comprise osteoclasts and, optionally, osteoblasts.
22. A method according to paragraph 20 or paragraph 21, wherein step iv) comprises assessing the resorption of the bone-like coating and the formation of bone.
23. A method according to any one of paragraphs 20 to 22, wherein the method comprises fluorescently labelling the bone-like coating.
24. A method according to paragraph 23, wherein step iv) comprises monitoring the fluorescence of the bone-like coating and/or the cell culture.
25. A method according to any one of paragraph 20 to 24, wherein the method is a method for assaying a pharmaceutical compound, the method comprising step iii): contacting the cell culture with a pharmaceutical compound.
26. A kit comprising: simulated body fluid (SBF), wherein the concentration of Ca²⁺ in the SBF is ≥7.5 mM and the concentration of HPO₄²⁻ in the SBF is ≥3 mM; and a collagen solution.
27. A kit according to paragraph 26, wherein the concentration of Ca²⁺ in the SBF is ≥18.8 mM and the concentration of HPO₄²⁻ in the SBF is ≥7.5 mM.
28. A kit according to paragraph 26 or paragraph 27, wherein the concentration of collagen in the collagen solution is in the range from 4 mg/ml to 8 mg/mL.
29. A kit according to any one of paragraph 26 to 28, wherein the kit further comprises sodium bicarbonate.
30. A coated substrate, comprising:
   a substrate; and
   a coating on the substrate, wherein the coating comprises calcium phosphate at the interface between the substrate and coating and collagen deposited on the calcium phosphate.
31. A coated substrate obtainable by any one of paragraphs 8-19.

### BRIEF DESCRIPTION OF THE DRAWINGS

Embodiments of the invention are further described hereinafter with reference to the accompanying drawings, in which:

FIG. 1 shows Brightfiled images of (A) coated TCP, (B) coated stereolithograpic 3D printed polymer and (C) representative close-up image of the coated surface.

FIG. 2 shows the mineral deposition on TCP during the coating process with investigated collagen concentrations (low magnification, scale bar 50 μm).

FIG. 3 shows the mineral deposition on TCP during the coating process with investigated collagen concentrations (high magnification, scale bar 3 μm).

FIG. 4 shows the cellular morphology after 1 day of culture on 10×SBF and 10× SBF collagen (1 mg/mL) coated TCP. (A) Live dead staining of cells cultured on TCP. (B) Live dead staining of cells cultured on 10×SBF mineralized TCP. (C) Live dead staining of cells cultured on collagen supplemented (1 mg/mL) 10×SBF mineralized TCP. (D) Lactate dehydrogenase (LDH) toxicity assay after 1 day. (E) Mean cell circularity and (F) mean cell surface area. Scale bar 200 μm. ANOVA and Bonferroni's multiple comparison test. LDH n>3 circularity n>80, area n>80.

FIG. 5 shows the cell viability on collagen supplemented (1 mg/mL) 10×SBF mineralized TCP surfaces. (A) Live dead staining of cells cultured on TCP after 1 day. (B) Live dead staining of cells cultured on 10×SBF coated TCP after 1 day. (C) Live dead staining of cells cultured 10×SBF collagen (1 mg/mL) coated TCP after 1 day. (D) Live dead staining of cells cultured on TCP after 7 days. (E) Live dead staining of cells cultured on 10×SBF coated TCP after 7 days. (F) Live dead staining of cells cultured 10×SBF collagen (1 mg/mL) coated TCP after 7 days. (G) Metabolic activity measurement on day 1,3 and 7 after seeing. ANOVA and Bonferroni's multiple comparison test n=3.

FIG. 6 shows the cell viability and morphology on 10×SBF collagen coated TCP with varying collagen concentrations after 1 day of culture. (A) Live dead staining of the TCP control. (B) Live dead staining of the 10×SBF 0.25 mg/mL collagen coated TCP surface. (C) Live dead staining of the 10×SBF 0.5 mg/mL collagen coated TCP surface. (D) Live dead staining of the 10×SBF 1 mg/mL collagen coated TCP surface. (E) LDH toxicity assay after 1 day. (F) Mean cell circularity and (G) mean cell surface area. ANOVA and Bonferroni's multiple comparison test, Cytotoxicity n=3, circularity n>72, area n>53.

FIG. 7 depicts the qualitative cell viability on collagen supplemented 10×SBF mineralized TCP surfaces with varying collagen concentrations over a 7-day culture period. (A) Live dead staining of the TCP control after 1 day. (B) Live dead staining of the 10×SBF 0.25 mg/mL collagen coated TCP surface after 1 day. (C) Live dead staining of the 10×SBF 0.5 mg/mL collagen coated TCP surface after 1 day. (D) Live dead staining of the 10×SBF 1 mg/mL collagen coated TCP surface after 1 day. (E) Live dead staining of the TCP control after 7 days. (F) Live dead staining of the 10×SBF 0.25 mg/ml collagen coated TCP surface after 7 days. (G) Live dead staining of the 10×SBF 0.50 mg/mL collagen coated TCP surface after 7 days. (H) Live dead after 7 days staining of the 10×SBF 1 mg/mL collagen coated TCP surface after 7 days.

FIG. 8 depicts the quantitative cell viability on collagen supplemented 10×SBF mineralized TCP surfaces over a 7-day culture period. (A) Metabolic activity assay. (B) DNA quantification assay after 7 days of culture. ANOVA and Bonferroni's multiple comparison test, metabolic activity n=3, DNA content n=3

FIG. 9 shows representative images of osteoblast osteoclast co-cultures on calcein green stained 10×SBF collagen (1 mg/mL) coating. (A) Phase contrast image of an osteoclast-osteoblast co-culture in a 1:7 ratio. (B) Respective field of view stained in calcein green. (C) Thresholded image to quantify osteoblast mediated minerals formation. (D) Thresholded image to quantify osteoclast mediated resorption of coating. (E) Quantification of cell mediated mineral formation and resorption. ANOVA and Bonferroni's multiple comparison test. Scale bar 100 μm.

FIG. 10: Von Kossa staining of 5×SBF 1 mg/mL collagen coated surfaces. TCP was incubated in an inverted orientation. (A) 2 hours (B) 4 Hours (C) 6 hours (D) overnight. Scale bar: 500 μm.

FIG. 11: Von Kossa staining of 5×SBF 1 mg/mL collagen coated surfaces. TCP was incubated in an upright orientation. (A) 2 hours (B) 4 Hours (C) 6 hours (D) overnight. Scale bar: 500 μm.

FIG. 12: Von Kossa staining of 10×SBF 1 mg/mL collagen coated surfaces. TCP was incubated in an inverted orientation. (A) 2 hours (B) 4 Hours (C) 6 hours (D) overnight. Scale bar: 500 μm.

FIG. 13: Von Kossa staining of 10×SBF 1 mg/mL collagen coated surfaces. TCP was incubated in an upright orientation. (A) 2 hours (B) 4 Hours (C) 6 hours (D) overnight. Scale bar: 500 μm.

FIG. 14: Von Kossa staining of 10×SBF collagen coated surface. (A) Surface incubated in H₂O at 37° C. overnight. (B) Surface incubated in DMEM at 37° C. overnight. Scale bar: 500 μm.

### DETAILED DESCRIPTION

A simulated body fluid (SBF) is a solution with an ion concentration similar to that of human blood plasma comprising, e.g. Nat, K⁺, Mg²⁺, Ca²⁺, Cl⁻, HCO₃⁻; and HPO₄²⁻ ions in specific concentrations. SBF is often labelled as n×SBF, wherein ‘n’ is a number and ‘n×’ denotes an increase in ion concentration relative to human blood plasma. For example, conventional SBFs (i.e., 1, 1.5, 2, or 5×SBF) have relatively low calcium and phosphate ion concentrations, namely, 2.5 mM and 1.0 mM, respectively, for 1×SBF. 10×SBF, which may be used in the formulations and methods of the present invention, has calcium and phosphate ion concentrations of 25 mM and 10 mM, respectively.

The term “bone-like coating” is intended to refer to a coating that has a similar chemical constituency to that of human bone. The ‘bone-like coating’ comprises calcium phosphate.

A “fluorescent label” is a compound that fluoresces when exposed to an appropriate wavelength of light. The term “fluorescent label” and “fluorophore” may be used interchangeably.

The term “fluorescently labelled bone-like coating” is intended to encompass a bone-like coating that is labelled with a fluorescent label. The term “label” denotes some form of chemical interaction between the fluorescent label and the bone-like coating. For example, the fluorescent label may interact with the bone-like coating via an intermolecular attraction or attractions, e.g. hydrogen bonding and/or Van der Waals forces. Alternatively, the fluorescent label may interact with the bone-like coating via the formation of covalent or dative bonds.

Tissue culture plastic (TCP) is polystyrene that has been plasma treated in order to increase cell adhesion. This partially modifies and cleaves the polymer chain, leaving behind oxygen-containing functional groups such as hydroxyl and carboxyl.

The term “calcium phosphate” is intended to cover calcium apatites, e.g. hydroxyapatite.

Atelocollagen is type I collagen that has had the telopeptide regions removed by protease treatment.

Basal media are culture media that can be used to grow (culture) bacteria without the need for additional media enrichment. Examples include nutrient broth, nutrient agar, peptone water and DMEM.

Cell culture plates typically comprise a well or wells. In the method of the second aspect of the invention, when the substrate is a cell culture plate, step c) may comprise submerging the cell culture plate in the coating formulation in an upright position or in an inverted position. In the upright position, the open face of the well or wells of the cell culture plate face away from the direction of gravity. In the inverted position, the open face of the well or wells of the cell culture plate face towards from the direction of gravity thereby allowing any precipitate that forms during step c) to gravitate out of the well or wells.

Throughout the description and claims of this specification, the words “comprise” and “contain” and variations of them mean “including but not limited to”, and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics, compounds, chemical moieties or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

### Examples

### Example 1: Coating (1 mg/mL Collagen)

A 10×SBF solution was prepared according to the method disclosed in A. C. Tas and S. B. Bhaduri, J. Mater. Res., 2004, 19, 2742-2749. The following reagents in Table 1 were obtained from Sigma-Aldrich and added to 1900 mL of deionized water under constant stirring at room temperature.

TABLE 1
Reagents for the preparation of 2 L 10× SBF stock solution.

| Order of | | Amount | Concentration |
|---|---|---|---|
| addition | Reagent | (g) | (mM) |
| 1 | NaCl | 116.8860 | 1000 |
| 2 | KCl | 0.7456 | 5 |
| 3 | CaCl₂•2H₂O | 7.3508 | 25 |
| 4 | MgCl₂•6H₂O | 2.0330 | 5 |
| 5 | NaH₂PO₄ | 2.3996 | 10 |

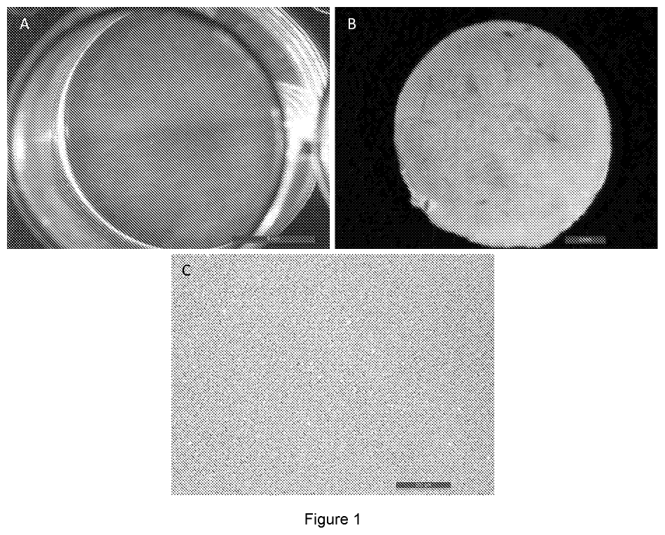

After all the reagents were added, the solution was topped up with deionized water to a volume of 2 L. 20 mL of this stock solution was added to a 50 mL glass beaker and 4 mL of 6 mg/mL atelocollagen solution (Pepsin Soluble Atelo Collagen in 0.01M HCl obtained from Collagen Solutions) was added. 30 mM of NaHCO₃ was added stepwise over 3 minutes in order to raise the pH of the solution to within the range of from 6.25-6.30 before the solution was filtered using a 100 μm filter. 3D printed objects or tissue culture plastic (TCP) were then incubated in this solution for up to 4 h. After 2 (TCP) and 4 (printed polymer) hours of incubation, the formation of a calcium phosphate coating on the surface of the substrate was observed (FIG. 1).

At the end of the incubation, the coated objects were removed from the solution, washed with deionized water and dried at room temperature. The surfaces of the coated objects were UV sterilised for 30 mins before cell culture experiments were started.

### Example 2: Coating (Varying Collagen Concentrations)

0.5, 0.25 and 0 mg/mL collagen solutions were prepared according to the process described above in which 2 mL, 1 mL and 0 mL, respectively, of atelocollagen solution was added to the stock solution.

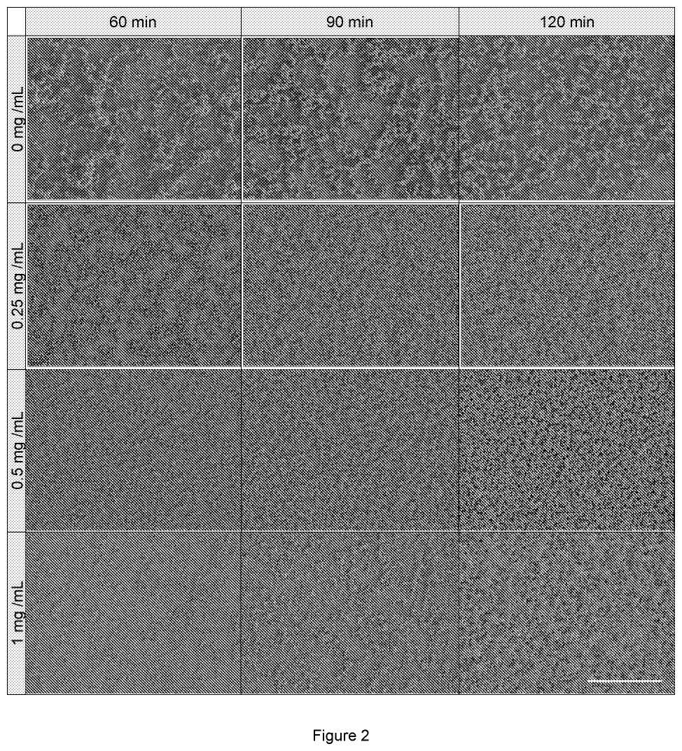

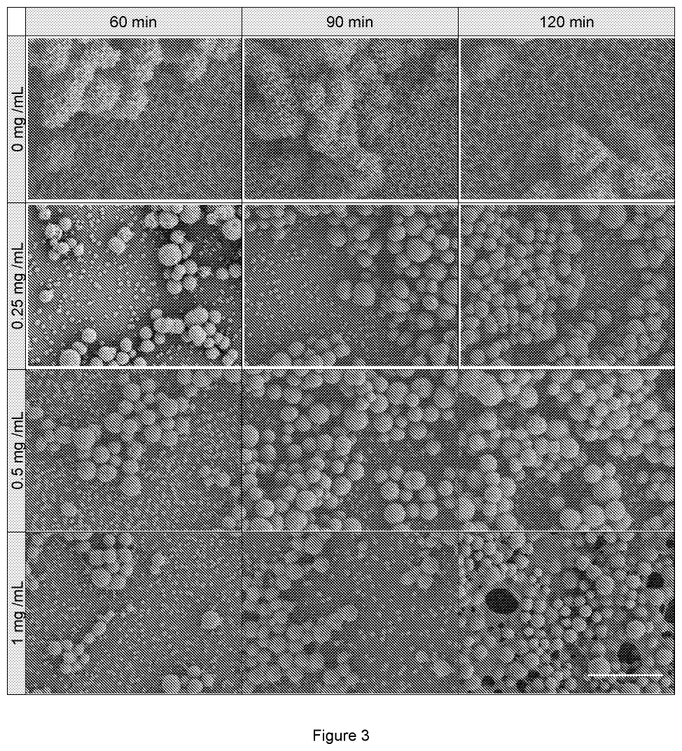

The images in FIG. 2 and FIG. 3 show the morphology of the coating by SEM for a coating period of 2 h with varying collagen concentrations. Comparison of the images shows that the coating becomes denser over the incubation period across all investigated collagen concentrations.

The high magnification images in FIG. 3 show that the 10×SBF coating without collagen shows a different morphology than the formulations with collagen. Without collagen the coating consists of a net-like structure on the bottom with bigger coral like particles on top. In contrast, the 0.25, 0.5 and 1 mg/mL collagen supplemented formulations form spherical particles on the surface. With increasing collagen concentration and incubation time more collagen is incorporated in the coating.

### Example 3: Cell Viability

The integration of atelocollagen into the existing 10×SBF coating process and its effect on cell viability was investigated.

hTERT-BMSCs Y201 cells were cultured and prepared for live dead staining on TCP and 10×SBF and 10×SBF collagen (1 mg/mL) coated TCP surfaces using the LIVE/DEAD® Viability/Cytotoxicity Kit for mammalian cells obtained from Thermo Fisher Scientific.

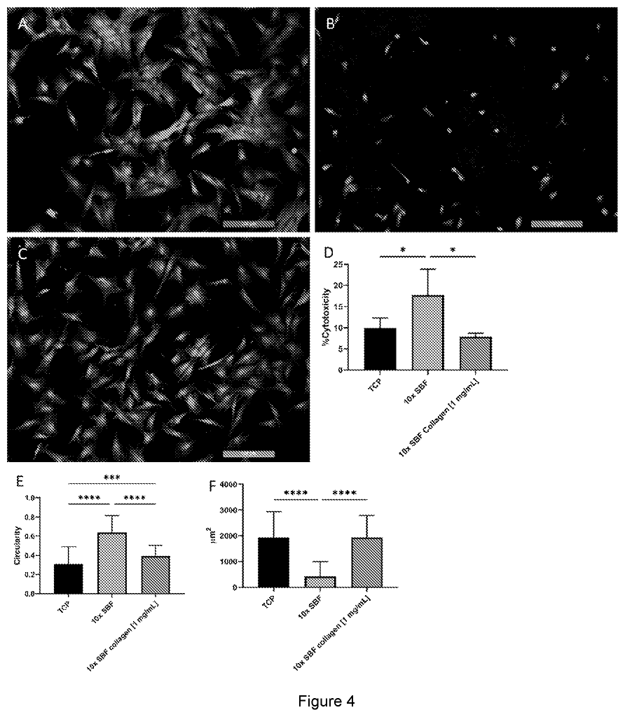

The live dead staining after 1 day (shown in FIGS. 4A, B and C) indicated a high cell viability on the TCP, 10×SBF and 10×SBF collagen (1 mg/mL) coated surfaces. Also, the LDH cytotoxicity assessment indicated a high viability on all 3 surfaces after 1-day post seeding (FIG. 4 D). The 10×SBF coating caused a higher cytotoxicity (18%) than the 10×SBF collagen coating (8%) and the TCP control (12%).

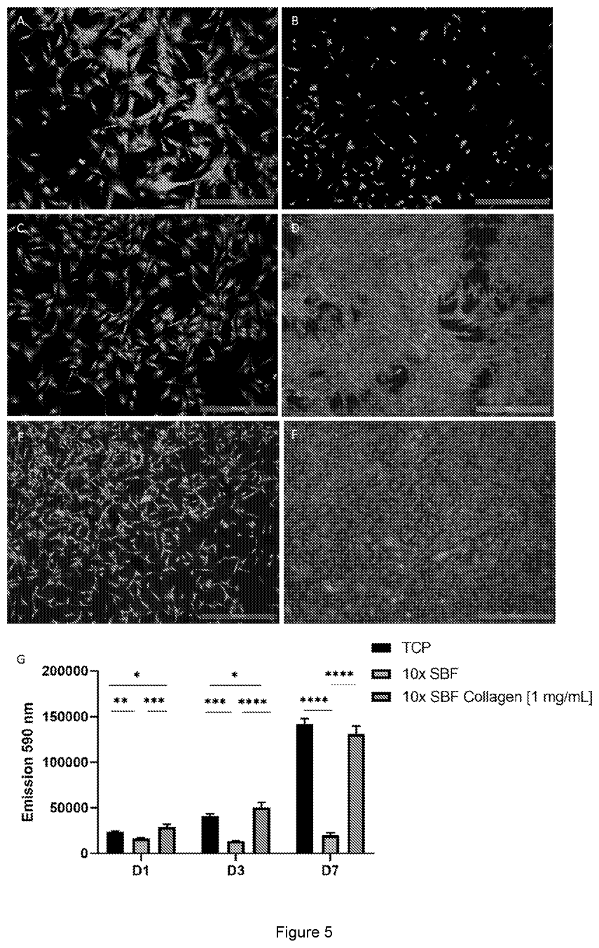

Cells that were cultured on the 10×SBF coated surface had a rounder morphology in comparison to cells on the TCP control after 1 day (FIG. 5A, B, E). Cells that were seeded on the 10×SBF collagen surface showed an elongated morphology similar to the TCP control (FIG. 4 C, E). The cell surface area of cells on the 10×SBF coated surface was significantly smaller than on the 10×SBF collagen and TCP (FIG. 4 F). However, there was no significant difference between cells cultured on 10×SBF collagen and TCP (FIG. 4 F).

The pictures of the live dead staining after 1 day show a low cell density on all three surfaces (FIGS. 5A, B and C) with some cell agglomerates in the TCP control (FIG. 5A). The live dead staining after 7 days of culture indicated a high viability and an increase in cell number on each surface (FIGS. 5 D, E and F). Cells on the TCP grew into a confluent monolayer (FIG. 5 D) while cells on the 10×SBF coating were less abundant and also showed relatively more red fluorescence signals indicating dead cells (FIG. 5 E). In contrast to the pictures on day 1, cells on the 10×SBF exhibited now a flattened cell morphology. The cells cultured on the 10×SBF collagen surface were abundant and growing into a confluent monolayer similar to the results observed on TCP (FIG. 5 F). Also, fewer dead cells were detected than in the 10×SBF group.

The measurement of the metabolic activity showed that cells seeded on the 10× SBF collagen surface had a comparable total activity to the TCP control indicating a similar number of viable cells (FIG. 5 F). Moreover, the activity in both groups increased between day 1 and day 7 portending an increasing cell number over the culture period. In contrast, the cells that were cultured on the 10×SBF surface had a relatively lower total metabolic activity on days 1, 3 and 7 and the activity increased only slightly during the 7 days of culture. Together, the live dead staining and the metabolic activity data suggest that cells on the 10×SBF collagen coating were proliferating on a similar level as on TCP while on the 10×SBF coating proliferation was slowed.

As these results indicated that the addition of collagen with a concentration of 1 mg/mL improved the cell viability it was investigated whether similar results could be obtained with decreased concentrations.

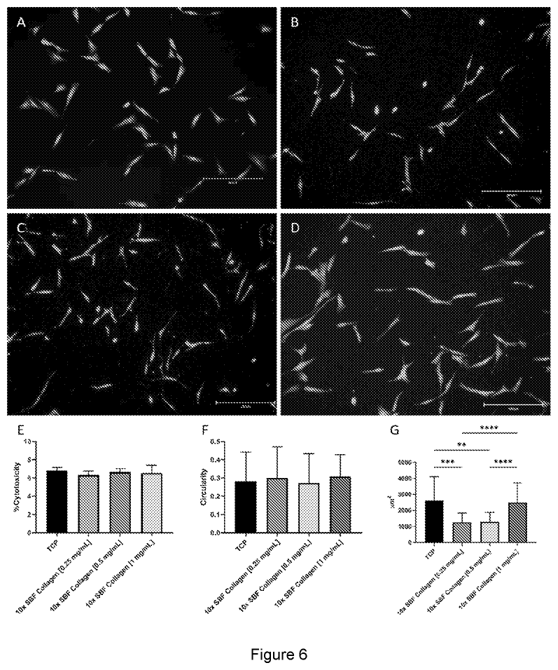

The cellular morphology after 1 day of seeding shows that cells cultured on 0.25 mg/ml and 0.5 mg/mL collagen (FIG. 6 B, C) were thin spindle shaped while cells on 1 mg/mL collagen in comparison had a spread morphology (FIG. 6 D), which was similar to that observed on TCP (FIG. 6A). All groups showed no or few signals for dead cells. The quantitative image analysis showed no differences in cell circularity (FIG. 6 F) but indicated significant differences in terms of the cell surface area (FIG. 6 G). Cells on the 1 mg/mL collagen coated surface spread on a comparable level as on TCP while the reduction 0.5 and 0.25 mg/mL collagen lead a significantly decreased cell surface area (FIG. 6 G). No significant differences between the coated surfaces in terms of LDH release 1-day post seeding were observed. All groups showed low values around 7% and were on level with the TCP control (FIG. 6 E).

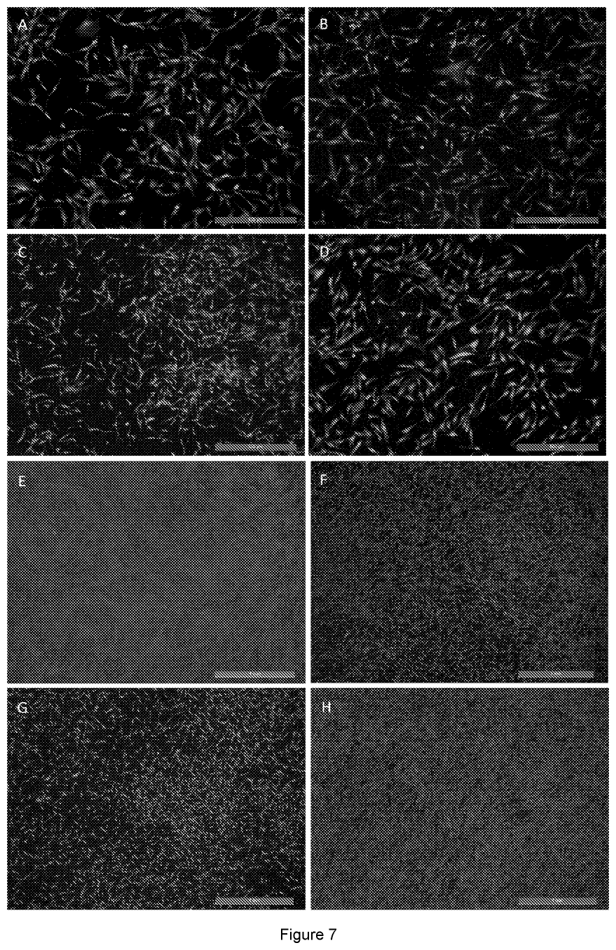

The live dead staining after 1 day showed a low cell density on all 4 surface types (FIG. 7A-D). The live dead staining after 7 days of culture indicated differences in the total cell number between the 4 experimental groups. Cells grown on TCP (FIG. 7 E) and 1 mg/mL collagen (FIG. 7 H) grew into a confluent monolayer. The cell numbers on 0.25 mg/mL (FIG. 7 F) and 0.5 mg/mL collagen (FIG. 7 G) were also increased but were lower than on TCP and 1 mg/mL. All groups showed few signals for dead cells on both time points indicating cell high viability.

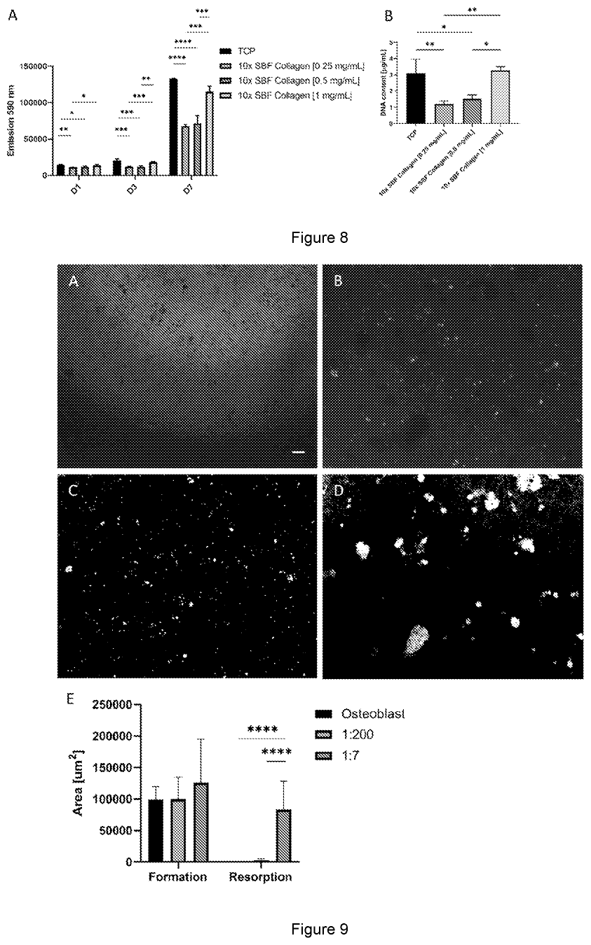

The measurement of the metabolic activity between day 1 and day 7 indicates an increasing activity and thus increasing number of viable cells in all groups during this time frame (FIG. 8A). At all 3 time points the activity was lowest in the 0.25 mg/ml and 0.5 mg/mL collagen group while the 1 mg/mL was on a comparable level to the TCP control. This data was further supported by DNA quantification assay after 7 days (FIG. 8 B). The 1 mg/mL collagen and TCP groups had the highest concentration of DNA and were significantly higher than 0.25 and 0.5 mg/mL group. Even though proliferation was slowed on the 0.25 and 0.5 mg/mL collagen 10×SBF coated surfaces it is important to note that cells on these surfaces proliferated relatively more than cells on plain 10×SBF surfaces.

### Example 4: Bone Remodelling Activity of Osteoblasts and Osteoclasts

Mesenchymal stem cells (MSCs) were cultured for 3-4 weeks in an osteogenic medium to allow differentiation into osteoblasts before being seeded onto tissue culture plastic coated using the 1 mg/mL collagen formulation. After 1 week of further culture, osteoclasts were added to the MSCs.

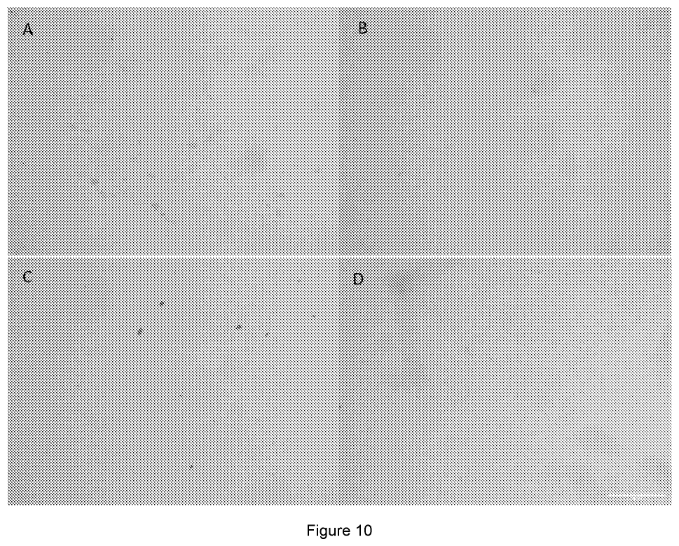

The bone remodelling activity of osteoblasts and osteoclasts was then investigated. The developed coating allows cell mediated mineral formation and resorption to be assessed and quantified. Using the fluorescent calcium binding dye calcein green it is possible to distinguish between the coating (light green), freshly formed minerals (bright green) and resorption pits (dark areas) in an osteoblast-osteoclast co-culture. Representative images are shown in FIG. 9.

The phase contrast image (A) shows that the coating allows cells to be visualised on TCP during the cell culture period. The respective image of the calcein stained coating (B) shows that resorption pits made by osteoclasts and freshly deposited minerals deposited by osteoblast can be visually separated on the coating. Quantification of the formed minerals (C) is achieved by using a high-intensity threshold on the captured image. Quantification of the resorbed minerals (D) is achieved by using a low-intensity fluorescence threshold on the captured image.

The quantification of the remodelling activity of osteoclast and osteoblast in a 1:7 and 1:200 ratio to each other (E) indicates that the resorbed area increases with an increasing number of osteoclasts on the coating while the area of freshly formed minerals remains unaffected by the number of osteoclasts in the co-culture.

A new biomimetic coating methodology for bone tissue engineering and disease modelling applications has therefore been developed which allows for the quantification of both osteoclast and osteoblast mediated formation and resorption.

### Example 5: 5×SBF vs. 10×SBF

The calcium phosphate deposition caused by the 5× and 10×SBF/collagen solutions was assessed.

A 5×SBF solution was prepared according to Table 2. The reagents (all from Sigma-Aldrich) were added to 1900 mL of deionized water under constant steering at room temperature.

TABLE 2
Reagents for the preparation of 2 L 5× SBF stock solution.

| Order of | | Amount | Concentration |
|---|---|---|---|
| addition | Reagent | (g) | (mM) |
| 1 | NaCl | 58.443 | 500 |
| 2 | KCl | 0.3728 | 2.5 |
| 3 | CaCl₂•2H₂O | 3.6754 | 12.5 |
| 4 | MgCl₂•6H₂O | 1.0165 | 2.5 |
| 5 | NaH₂PO₄ | 1.1998 | 5 |

After all the reagents were added, the solution was topped up with deionized water to a volume of 2 L. Prior to sample coating 21 mL of this stock solution was added to a 50 ml capacity glass beaker. 4 mL of a 6 mg/mL atelocollagen solution was then added. 30 mM of NaHCO₃ was then added stepwise over 3 minutes to raise the pH of the solution to around 6.25-6.30. After 3 minutes the solution was filtered using a 100 μm filter.

A 10×SBF solution comprising 1 mg/mL collagen was prepared as outlined in Example 1, above.

96 well cell culture plates (made from TCP) were incubated in each solution in upright and inverted orientations for 2 h, 4 h, 6 h or overnight (18 h) at room temperature. At the end of the incubation period, the well plates were taken out of solution, washed with deionized water and dried at room temperature. The von Kossa staining technique was used to stain any calcium phosphate deposited on the surface of the well plates. Images are shown in FIG. 10-13.

The images of the inverted coated surfaces show that there was sparse deposition of calcium phosphates using the 5×SBF 1 mg/mL collagen solution after 2 h, 4 h, 6 h and overnight incubation (FIG. 10).

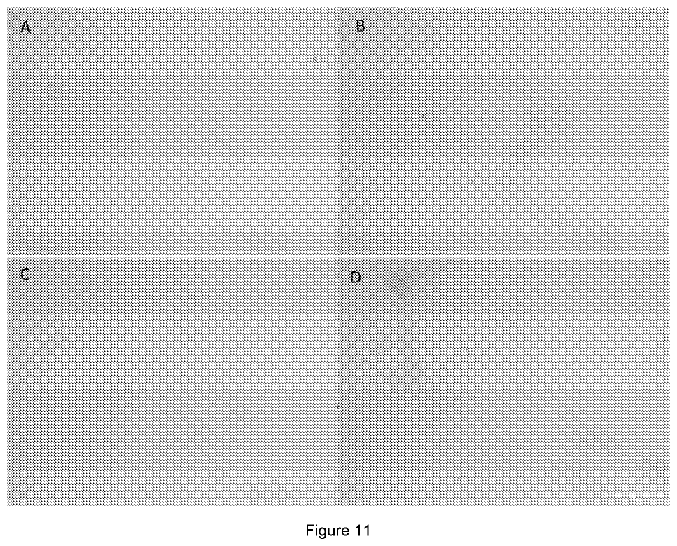

The images of the upright coated surfaces show that there was a sparse deposition of calcium phosphates using the 5×SBF 1 mg/mL collagen solution after 2 h, 4 h, 6 h and overnight incubation (FIG. 11).

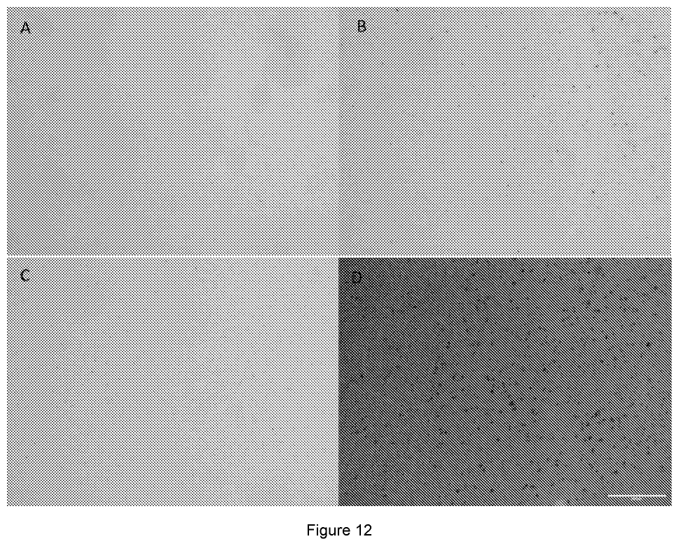

The images of the inverted coated surfaces using the 10×SBF 1 mg/mL collagen solution after 2 h, 4 h, 6 h and overnight incubation are shown in FIG. 12. FIG. 12D illustrates that a homogeneous calcium phosphate layer was deposited following an overnight incubation, whereas minimal deposition was observed after 2-6 hours.

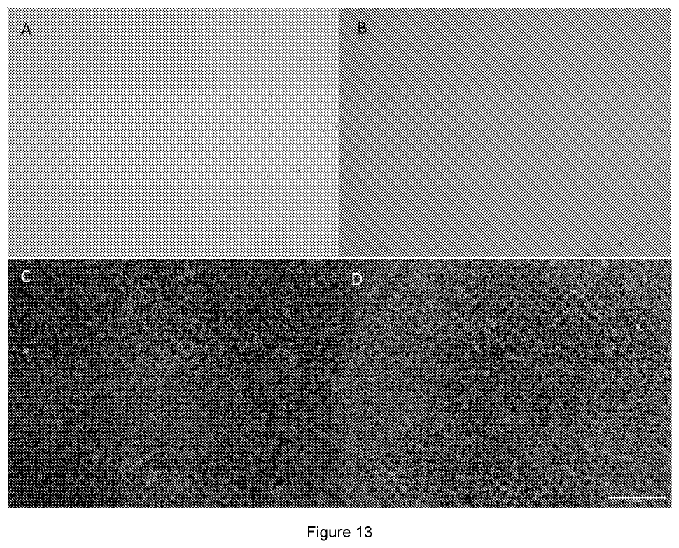

FIG. 13 showcases a series of images featuring upright coated surfaces that have undergone 2-hour, 4-hour, 6-hour, and overnight incubation using the 10×SBF 1 mg/mL collagen solution. The von Kossa staining demonstrates a strong deposition of calcium phosphates after 6 h and overnight incubation.

The inventors of the present invention have surprisingly found that the use of a 10×SBF/collagen solution allows for the quicker deposition of apatite on the surface of a substrate, even at lower temperatures, than a 5×SBF/collagen solution. The formulations of the present invention therefore provide a practical and resource efficient means for forming a bone-like coating on the surface of a substrate.

### Example 6: DMEM Incubation

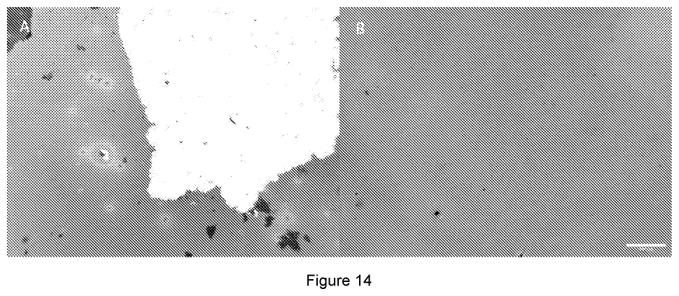

The effect of overnight incubation in Dulbecco's Modified Eagle's Medium (DMEM), a basal medium, was studied. Coated 96 well plates were prepared according to Example 1, above. 100 μL of DMEM was added to each well of a coated 96 well plate. 100 μL of water was added to each well of another coated 96 well plate. Each plate was incubated overnight (18 h) at 37° C. The von Kossa staining technique was used to stain calcium phosphate on the coated surfaces (FIG. 14).

The results show that the coating detached in wells that were incubated in water whereas the coating in the wells that were incubated with DMEM further adhered to the surfaces. Without wishing to be bound by theory, it is thought that the DMEM solution leads to further crystallization of the deposited calcium phosphates thereby increasing the stability of the bon-like coating.

1-32. (canceled)

33. A coating formulation comprising simulated body fluid (SBF) and collagen, wherein the concentration of Ca²⁺ in the formulation is >12.5 mM and the concentration of HPO₄²⁻ in the formulation is >5.0 mM.

34. The formulation according to claim 33, wherein the concentration of Ca²⁺ in the formulation is ≥18.8 mM and the concentration of HPO₄²⁻ in the formulation is ≥7.5 mM.

35. The formulation according to claim 33, wherein the concentration of collagen in the formulation is in the range from 0.1 mg/ml to 5.0 mg/mL.

36. The formulation according to claim 35, wherein the concentration of collagen in the formulation is in the range from 0.25 mg/ml to 0.45 mg/mL.

37. The formulation according to claim 33, wherein the pH of the formulation is in the range from 6.1 to 6.6.

38. The formulation according to claim 37, wherein the pH of the formulation is in the range from 6.25 to 6.30.

39. The formulation according to claim 33, wherein the formulation further comprises sodium bicarbonate.

40. A method for depositing a bone-like coating onto a substrate, the method comprising step c):
c) contacting the substrate with a coating formulation according to claim 33 to form a coated substrate.

41. The method according to claim 40, wherein the method further comprises steps a) and b):
a) adding collagen to the simulated body fluid (SBF), wherein the concentration of Ca²⁺ in the SBF is >12.5 mM and the concentration of HPO₄²⁻ in the SBF is >5.0 mM, to form an intermediate formulation; and
b) adjusting the pH of the intermediate formulation to be in the range from 6.1 to 6.6 to form the coating formulation.

42. The method according to claim 41, wherein the concentration of collagen in the collagen solution is in the range from 4 mg/ml to 8 mg/mL.

43. The method according to claim 41, wherein step b) further comprises adjusting the pH of the intermediate formulation to be in the range from 6.25 to 6.30.

44. The method according to claim 41, wherein step b) further comprises adding sodium bicarbonate to the intermediate formulation.

45. The method according to claim 41, wherein the concentration of Ca²⁺ in the SBF is ≥18.8 mM and the concentration of HPO₄²⁻ in the SBF is ≥7.5 mM.

46. The method according to claim 41, wherein the pH of the SBF is in the range from 6.1 to 6.6.

47. The method according to claim 40, wherein the substrate is a metal substrate.

48. The method according to claim 40, wherein the substrate is a polymer substrate.

49. The method according to claim 48, wherein the substrate is a cell culture plate.

50. The method according to claim 40, wherein the substrate is a 3D printed object and/or a medical implant.

51. The method according to claim 40, wherein the method further comprises step d): incubating the coated substrate in a basal medium for at least 6 hours.

52. A method comprising steps i), ii) and iv):
i) carrying out a method according to claim 40 to form a coated substrate;
ii) culturing cells on the coated substrate to form a cell culture; and
iv) monitoring the bone-like coating and/or bone remodeling activity of the cell culture.

53. The method according to claim 52, wherein the cells comprise osteoclasts and, optionally, osteoblasts.

54. The method according to claim 52, wherein step iv) further comprises assessing the resorption of the bone-like coating and the formation of bone.

55. The method according to claim 42, wherein the method further comprises fluorescently labelling the bone-like coating.

56. The method according to claim 55, wherein step iv) further comprises monitoring the fluorescence of the bone-like coating and/or the cell culture.

57. The method according to claim 52, wherein the method is a method for assaying a pharmaceutical compound, and wherein the method further comprises step iii): contacting the cell culture with a pharmaceutical compound.

58. A kit comprising:
simulated body fluid (SBF), wherein the concentration of Ca²⁺ in the SBF is >12.5 mM and the concentration of HPO₄²⁻ in the SBF is >5.0 mM; and
a collagen solution.

59. The kit according to claim 58, wherein the concentration of Ca²⁺ in the SBF is ≥18.8 mM and the concentration of HPO₄²⁻ in the SBF is ≥7.5 mM.

60. The kit according to claim 58, wherein the concentration of collagen in the collagen solution is in the range from 4 mg/ml to 8 mg/mL.

61. The kit according to claim 58, wherein the kit further comprises sodium bicarbonate.

62. A coated substrate, comprising:
a substrate; and
a coating on the substrate, wherein the coating comprises calcium phosphate at the interface between the substrate and coating and collagen deposited on the calcium phosphate.

63. A coated substrate obtained by a method according to claim 40.
